(19) United States
(12) Patent Application Publication
(10) Pub. No.: US 2010/0272765 A1
(43) Pub. Date: Oct. 28, 2010

(54) Stable emulsion and process for preparing the same

(75) Inventors: Boen Ho O, Utrecht (NL); Jozef Johannes Maria Baltussen, Nijmegen (NL)

Correspondence Address: AKZO NOBEL INC., LEGAL & IP, 120 WHITE PLAINS ROAD, SUITE 300, TARRYTOWN, NY, 10591, US

(73) Assignee: AKZO NOBEL N.V., Arnhem (NL)

(21) Appl. No.: 12/811,955

(22) PCT Filed: Jan. 8, 2009
(86) PCT No.: PCT/EP2009/050185

§ 371 Date: Jul. 7, 2010

Related U.S. Application Data
(60) Provisional application No. 61/022,699, filed on Jan. 22, 2008.

(30) Foreign Application Priority Data
Jan. 9, 2008 (EP) 08100273.5

(51) Int. Cl. A61K 8/06; A01N 25/04

(52) U.S. Cl. 424/401; 424/405

(57) ABSTRACT

The invention relates to a stable emulsion comprising (a) an oil; (b) water; (c) a surfactant; and (d) solid particulate material, wherein an emulsion comprising the oil, water, and the surfactant has a transitional phase inversion point, wherein the stable emulsion contains droplets having a d50 value of below 1 μm, and wherein the shelf life time of an emulsion comprising compounds (a) to (d) is longer than the shelf life time of emulsions containing only compounds (a) to (c).

The invention relates to emulsions comprising (a) an oil; (b) water; and (c) a surfactant.

Such emulsions are known in the art and are commonly referred to as oil-in-water or water-in-oil emulsions. Also, such emulsions generally have a limited stability, i.e. limited storage life time or shelf life time, and segregate or separate upon prolonged storage, and/or show rapid droplet growth or droplet size increase.

In the context of the present invention the term “storage life time” or “shelf life” refers to the period up to incipient separation, and in which the emulsion does not visually show segregation, such as the formation of a visible bottom layer of water and/or a visible top layer of oil.

Within the group of emulsions defined above it is known that there are particular emulsion compositions showing a Transitional Phase Inversion (TPI). The TPI phenomenon is an intrinsic property of the emulsion and is revealed when preparing the emulsion. The TPI is in strong contrast to the Catastrophic Phase Inversion, which requires the use of a particular process, as is well-known in the art. A special type of TPI is the Phase Inversion Temperature (PIT). Some PIT emulsions spontaneously show a phase inversion under just mild agitation when subjected to a temperature change. More details on these phenomena can be found in, for instance, J. L. Salager, Adv. Colloid Interface Sci., 108-109, 259 (2004)).

It is known that an improved emulsion storage life time, and/or reduced droplet growth can be obtained if the TPI phenomenon is applied to prepare the emulsion. When the emulsion has a PIT, a temperature cycle provides the improved emulsion shelf life time. See, for instance, Th. Förster, F. Schambil W. von Rybinski, J. Dispersion Sci. Technol. 13, 183 (1992). However, even when making use of these effects, the emulsion storage life time may still be insufficient, in particular at elevated temperatures.

Hence, it is an object of the present invention is to provide a novel emulsion which has a good storage life time and/or thermal stability, i.e. a higher resistance to droplet size increase at elevated temperatures.

This object is achieved by providing a stable emulsion comprising (a) an oil; (b) water; (c) a surfactant; and (d) solid particulate material, wherein an emulsion comprising just the oil, water, and the surfactant has a TPI, wherein the stable emulsion contains droplets having a d50 value of below 1 μm, wherein the emulsions comprising (a) to (d) has a better shelf life time than emulsions containing only compounds (a) to (c) and wherein preferably the change in d50 value of the emulsion comprising compounds (a) to (d) is lower than the d50 value of emulsions containing only compounds (a) to (c) after prolonged storage.

It is noted that US 2006/057170 discloses that emulsions with particles can be produced if a specific choice of surfactants, oils, particles, and a specific oil/surfactant ratio is used. The amount of surfactant that is used is too high to be practical and only with specific oils, having a high molecular weight, can the emulsions be made.

Hence it is a further object of the invention to provide stable emulsions which have a low amount of surfactants and can be used for lower molecular weight oils.

It was surprisingly found that the solid particulate material enhances the storage life time of the emulsion when used in an emulsion whereof the oil, water, and the surfactant has a TPI, preferably a PIT. The presence of this phenomenon and the use of the solid particles causes the emulsion to have more resistance to droplet growth, and thus prevents coalescence and eventually segregation or separation. Hence, products of the invention show a better shelf life time than products of the prior art in which no solids are present and/or which have no TPI.

When compared to a similar emulsion in which the particulate material is absent, the emulsion of the invention preferably exhibits a smaller change in d50 value of the droplets, or no change in d50 value at all after prolonged storage.

For evaluating the storage stability/shelf life time, as used in this invention, a test method is to be used wherein a sample of 100 g of emulsion is stored in a test tube with an inner diameter of 2.5 cm and sufficient length. The tube is stored at a selected temperature and monitored over time for separation to occur, i.e. for formation of a top or bottom layer. The shelf life time is then the time elapsing between filling the test tube and the observation of the separation phenomenon. The temperature is to be chosen such that it is above the melting temperature of the compound in the emulsions with the highest melting temperature, and below the boiling temperature of the lowest boiling compound of the emulsion. Suitably it is chosen between 0° C. and 90° C.

In order to save time, the temperature is preferably chosen such that an emulsion comprising only compounds (a) to (c) shows separation within 2 weeks. In an embodiment of the invention, the emulsion in which solid particulate (d) is present has a shelf life time that is twice as long as the shelf life time of the emulsion containing only compounds (a) to (c) at a temperature wherein the latter emulsion has a shelf life time of less than 2 weeks. In further embodiments, the shelf life time is 4 or 10 times longer for the emulsions comprising the solid particulate (d).

It is noted that the effect found for the emulsions of the invention is contrary to the typical destabilizing effect of particles on emulsions. It is further noted that in another embodiment of the invention, the emulsion is not an oil in water emulsion whereof at least 25% by weight of the oil has a molecular weight greater than 400 Dalton and an oil/surfactant ratio is 0.8-3.5.

In an embodiment of the invention, the emulsion has an increased thermal stability, which implies that the stable emulsion has a higher resistance to droplet size increase at elevated temperatures, particularly at a temperature of about 50° C., compared to the same emulsion containing only compounds (a) to (c).

In compositions according to the invention it was found that the presence of the solid particulate material typically causes the emulsion of the invention to have a less pronounced phase inversion temperature, or to have a higher phase inversion temperature compared to emulsions containing only compounds (a) to (c). In an embodiment of the invention, the PIT of the emulsion of the invention typically is above the application temperature (i.e. the temperature at which the emulsion is applied) and/or above the storage temperature. In a particular embodiment of the invention, the PIT is above 30° C., preferably above 50° C., and most preferably above 70° C. Especially for oil in water emulsions the advantage of emulsions having a higher PIT is their use in a wider variety of applications, in particular applications conducted at higher temperatures. Moreover, emulsions having a higher phase inversion temperature generally have a higher thermal stability and/or a longer storage life time.

In case of emulsions with non-PIT TPI, the correlation between the composition at the TPI point and the shelf life time is comparable, namely that for o/w emulsions wherein the TPI is observed at a lower amounts of hydrophilics the stability is higher and that for w/o emulsions the stability was found to be higher for emulsions wherein the TPI is observed at a lower amounts of hydrophobics.

In one embodiment of the invention, the stable emulsion containing compound (d) has a droplet size that remains unchanged or is hardly increased upon segregation. These emulsions can be homogenized by shaking or (re-) stirring the segregated emulsion so as to form a uniform emulsion in which segregation is absent. Such emulsions thus can be stored (and then be allowed to segregate), and, subsequently, used in suitable applications after homogenization.

The emulsions of the invention can be oil-in-water (o/w) emulsions or water-in-oil emulsions (w/o). By “oil-in-water” emulsion is meant an emulsion where oil is dispersed as droplets and water is the continuous phase. By “water-in-oil” emulsion is meant an emulsion where water is dispersed as droplets and oil is the continuous phase. In the context of the present application the term “dispersed phase” refers to the droplets in the emulsion.

The droplets in the emulsion of the invention generally have a particle size distribution having a d50 value of below 1 μm. Preferably, the droplets have a d50 value of below 800 nm, more preferably below 600 nm, even more preferably below 500 nm, and most preferably below 300 nm.

The droplets in the emulsion generally have a particle size distribution having a d90 value of below 5 μm. Preferably, the droplets have a d90 value of below 2 μm, more preferably below 1 μm, even more preferably below 0.5 μm, and most preferably below 0.3 μm. The particle size distribution can be determined using methods known to the man skilled in the art, i.e. laser diffraction in accordance with DIN 13320.

The emulsions of the invention generally are liquid. It is also envisaged that the emulsion is a gel. However, liquid emulsions are preferred. In one embodiment the emulsions of the invention have a relatively low viscosity and are pourable. In another embodiment of the invention, the viscosity of the emulsions of the invention is below 10 Pa.s at a shear rate of 1,000 s⁻¹.

The emulsions of the invention contain water and oil. The oil generally is a hydrophobic phase which can comprise a wide variety of hydrophobic compounds known in the art. Examples of such oils or hydrophobic compounds include mineral oils including petrolatum; straight and branched chain hydrocarbons having from 7 to 40 carbon atoms such as dodecane, isododecane, squalane, cholesterol, hydrogenated polyisobutylene, isododecosane, hexadecane; C₁-C₃₀ alcohol esters of C₁-C₃₀ carboxylic acids and of C₂-C₃₀ dicarboxylic acids such as isononyl isononanoate, methyl isostearate, ethyl isostearate, diisoproyl sebacate, diisopropyl adipate, isopropyl myristate, isopropyl palmitate, methyl palmitate, myristyl propionate, 2-ethylhexyl palmitate, isodecyl neopentanoate, di(2-ethylhexyl) maleate, cetyl palmitate, cetyl stearate, methyl stearate, isopropyl stearate, and behenyl behenate; mono-, di-, and tri-glycerides of C₁-C₃₀ carboxylic acids such as caprylic/capric triglyceride, PEG-6 caprylic/capric triglyceride, and PEG-8 caprylic/capric triglyceride; alkylene glycol esters of C₁-C₃₀ carboxylic acids including ethylene glycol mono- and di-esters of C₁-C₃₀ carboxylic acids and propylene glycol mono- and di-esters of C₁-C₃₀ carboxylic acids such as ethylene glycol distearate; C₁-C₃₀ mono- and poly-esters of sugars and related materials such as glucose tetraoleate; and organopolysiloxane oils such as polyalkyl siloxanes, cyclic polyalkyl siloxanes, and polyalkylaryl siloxanes. Further specific examples of suitable oils are disclosed in US 2003/0228339. It is also contemplated to use propoxylated or ethoxylated forms of the above-exemplified oils. It is further envisaged to use two or more oils as the oil component in the emulsion of the invention.

In an embodiment of the invention the molecular weight of the oil is below 399, preferably below 390, more preferably below 350 and most preferably below 300 Dalton. In another embodiment essentially all oil in the emulsion has a molecular weight of the oil is below 399, preferably below 390, more preferably below 350 most preferably below 300 Dalton. Essentially all means, in this respect, that at most 20% by weight, suitably at most 10% by weight of all oil, has a higher molecular weight than indicated. Emulsions of such low molecular weight oils, water and surfactants, were found to often have a transitional phase inversion point and such emulsions were found to benefit from the addition of particles.

When the emulsion is a water-in-oil emulsion, the amount of water is at least 0.1 percent by weight (wt %), preferably at least 1 wt %, more preferably at least 15 wt %, and most preferably at least 30 wt %, and generally at most 99 wt %, preferably at most 90 wt %, more preferably at most 80 wt %, and most preferably at most 70 wt %, based on the total weight of the emulsion. The amount of oil in such emulsions is at least 0.1 wt %, preferably at least 1 wt %, more preferably at least 15 wt %, and most preferably at least 40 wt %, and generally at most 99 wt %, preferably at most 90 wt %, more preferably at most 80 wt %, and most preferably at most 70 wt %, based on the total weight of the emulsion. The total weight of oil and water in the emulsion generally is at most 99 wt %, preferably at most 95 wt %, and most preferably at most 85 wt %, based on the total weight of the emulsion.

When the emulsion is an oil-in-water emulsion, the amount of oil is at least 0.1 percent by weight (wt %), preferably at least 1 wt %, more preferably at least 15 wt %, and most preferably at least 30 wt %, and generally at most 99 wt %, preferably at most 90 wt %, more preferably at most 80 wt %, and most preferably at most 70 wt %, based on the total weight of the emulsion. The amount of water in such emulsions is at least 0.1 wt %, preferably at least 1 wt %, more preferably at least 15 wt %, and most preferably at least 40 wt %, and generally at most 99 wt %, preferably at most 90 wt %, more preferably at most 80 wt %, and most preferably at most 70 wt %, based on the total weight of the emulsion. The total weight of oil and water in the emulsion generally is at most 99 wt %, preferably at most 95 wt %, and most preferably at most 85 wt %, based on the total weight of the emulsion.

The surfactant which can be suitably used in the emulsions of the invention can be any surfactant known in the art as long as the emulsion containing water, oil, and the surfactant has a transitional phase inversion point. The surfactant can be an anionic, zwitterionic or amphoteric, nonionic or cationic surfactant, or a mixture of two or more of these surfactants. In an embodiment of the invention, a mixture of surfactants is used. In particular, mixtures of one or more anionic surfactants and one or more cationic surfactants, mixtures of one or more anionic surfactants and/or cationic surfactants and one or more nonionic surfactants, or mixtures of one or more amphoteric surfactants and one or more nonionic surfactants are envisaged. When the emulsion has a PIT, preferably, the surfactant is predominantly (>50% by weight) nonionic, and most preferably the surfactant is essentially (>90% w/w) non-ionic. Examples of suitable anionic surfactants include carboxylates, sulfates, sulfonates, phosphonates, and phosphates. Examples of suitable nonionic surfactants include alcohol ethoxylates, alkyl phenol ethoxylates, fatty acid ethoxylates, sorbitan esters and their ethoxylated derivatives, ethoxylated fats and oils, amine ethoxylates, ethylene oxide-propylene oxide copolymers, surfactants derived from mono- and polysaccharides such as the alkyl polyglucosides, and glycerides. Examples of suitable cationic surfactants include quaternary ammonium compounds. Examples of zwitterionic or amphoteric surfactants include N-alkyl betaines or other surfactants derived from betaines. More examples of specific surfactants can be found in US 2003/0228339.

The amount of surfactant used in the process of the invention is between 0.1 and 100 percent by weight (wt %), based on the total weight of the dispersed phase. Preferably, the amount is at least 0.5 wt %, more preferably at least 1 wt %, and most preferably at least 2 wt %, and preferably at most 30 wt %, more preferably at most 20 wt %, and most preferably at most 10 wt %, based on the total weight of the dispersed phase. In another embodiment the amount of surfactant is at most 30%, more preferably at most 20 wt %, and most preferably at most 10 wt %, of the weight of the oil in the emulsion.

In a further embodiment the amount of surfactant is chosen such that at least some surfactant is not associated with the surface of the solid dispersed phase, as will be the case if the above minimum amount of surfactant is used. This prerequisite sets the present emulsions apart from conventional particle-stabilized emulsions wherein all surfactant is associated with said surface.

The solid particulate material of the emulsion of the invention can be any solid particulate material known in the art which is suitable to enhance the storage lifetime and thermal stability of the emulsion of the invention. The solid particulate material can be cationic and/or anionic. Particularly suitable solid particulate materials are synthetic polymers and inorganic oxygen-containing particulate material, the latter being preferred. These materials include silica, bismuth oxychloride, titanated mica, phyllosilicates such as bentonite, hectorite or laponite; layered double hydroxides such as hydrotalcite or hydrotalcite-like materials; metal oxides such as iron oxide, magnesium oxide, titanium dioxide, zinc oxide, and alumina; calcium carbonate, magnesium carbonate, barium carbonate, barium sulfate, aluminium trihydroxide, calcium hydroxide, calcium acetate, calcium stearate, talc, glass, tricalcium phosphate, mica; and synthetic polymers such as polyethylene, polystyrene, polypropylene, acrylate polymers, and polymethyl methacrylate. More specific examples can be found in US 2003/0228339. Of these solid particulate materials silica is particularly suitable, in particular colloidal silica and fumed silica.

These solid particulate materials generally have an average particle size which is at least five times lower (i.e. the particles are five times smaller on average than the droplet size), preferably at least 10 times lower, and most preferably at least 20 times lower than the average droplet size. Typically, the d50 value of the solid particulate material is at most 200 nm, preferably at most 150 nm, and most preferably at most 100 nm, and generally at least 5 nm, preferably at least 10 nm, and most preferably at least 20 nm. In another embodiment the solid particles have at least in one dimension, preferably in two dimension, and most preferably in three dimensions, a size that is at least five times lower than the size of the droplets of the emulsion.

The amount of solid particulate material used in the process of the invention is between 0.1 and 100 percent by weight (wt %), based on the total weight of the dispersed phase. Preferably, the amount is at least 0.5 wt %, more preferably at least 1 wt %, and most preferably at least 2 wt %, and preferably at most 95 wt %, more preferably at most 50 wt %, more preferably at most 20 wt %, and most preferably at most 10 wt %, based on the total weight of the dispersed phase. It is noted that the weight of the dispersed phase is excluding any surfactant that is present on the surface of the dispersed phase and excludes any material that has gone in solution.

The invention further pertains to a process for preparing a stable emulsion comprising the steps of:
(a) contacting water, oil, and a surfactant so as to obtain an emulsion having a transitional phase inversion; and
(b) causing the emulsion to have a phase inversion so as to form a stable emulsion comprising droplets having a d50 value of below 1 μm; and
   wherein a solid particulate material is added to the emulsion before, during and/or after the phase inversion of step (b).

The phase inversion of step (b) can be invoked either by changing the composition of the emulsion, for instance by changing the oil to water ratio so as to cause a phase inversion—for example, adding water to a water-in-oil emulsion will lead to a reduction in the oil to water ratio, eventually resulting in an oil-in-water emulsion—and/or by changing the temperature—for example, by increasing the temperature to above the phase inversion temperature and subsequently lowering it to below the phase inversion temperature. The phase inversion step allows the conversion of an emulsion with droplets having a d50 value (well) above 1 μm to an emulsion with droplets having a d50 of below 1 μm. The process of the invention comprising such a phase inversion step is considerably less complex, easy to perform, requires less energy, and is economically more attractive compared to conventional processes for reaching emulsion with droplet sizes of a d50 of below 1 μm.

The invention further pertains to a process as described above wherein the phase inversion of step (b) is caused by heating the emulsion above its phase inversion temperature and subsequently lowering the temperature of the emulsion below the phase inversion temperature.

Alternatively, the phase inversion can be invoked by a change in composition. As exemplified above, this phase inversion can proceed by adding water to an emulsion so as to change the water to oil ratio. The phase inversion may also proceed by the addition of a surfactant to the emulsion or by the addition of a solution of surfactant and water to a solution of oil optionally containing another surfactant. It is also envisaged to start from an emulsion and add one or more of the ingredients of that emulsion so as to change the composition and cause a phase inversion. Subsequently, further ingredients are added to restore the composition of the initial emulsion or to obtain an emulsion with a different composition. In any case, the droplet size of the dispersed phase will be below 1 μm after the phase inversion.

It is also envisaged to prepare the emulsions of the invention using other methods known in the art to reduce the droplet size to a d50 value of below 1 μm, e.g. vigorous mixing in a conventional mixing apparatus. However, these conventional methods are less preferred because of their higher energy requirement and their complexity in comparison to processes comprising a phase inversion step.

The emulsions obtained in step (a) of the process of the invention have a phase inversion temperature. The phase inversion temperature generally is above the melting temperatures of water and the oil. Typically, the phase inversion temperature is above room temperature.

The emulsions of the invention can be used in any application for which they are suitable. Examples of such applications include use in cosmetics, drilling, oil recovery, food, agricultural chemicals, emulsion polymers or latexes, pharmaceuticals, and asphalt emulsions or asphaltic bitumen emulsions. Depending on the use of the emulsion, it can comprise further ingredients, which may either be oil-soluble or water-soluble. For instance, when used in agro formulations, the emulsion suitably contains an agro-chemically active compound. This can be the oil itself or any substance dissolved in the emulsion, such as biocides (including herbicides, fungicides, and pesticides), fertilizers, and the like. Said substance, or each substance when using a combination of substances, can be dissolved in any one of both phases. Similarly, e.g. for cosmetics, the emulsions can contain one or more additional compounds, such as the haloalkynyl compounds of US 2003/073689, perfumes, vitamins, and the like, dissolved in one or both phases, or as the oil component itself.

The invention is illustrated in the following examples.

### EXAMPLES

### Examples 1 and 2 and Comparative Example A

At room temperature an oil-in-water emulsion comprising a methyl ester of rape-seed oil, demineralized water, Agrilan® AEC 145, a di/tristyrylphenol ethoxylate (15EO) ex AkzoNobel, and sodium bis(2-ethylhexyl) sulfosuccinate was prepared in three steps as illustrated in the Table 1 below.

TABLE 1

| | step I | Step II | Step III |
|---|---|---|---|
| Ingredients | (g) | (g) | (g) |
| Methyl ester of rape-seed oil | 46 | | |
| Sodium bis(2-ethylhexyl) sulfosuccinate | 0.46 | 3.54 | |
| Water | | 49 | 147.55 |
| Agrilan ® AEC 145 | | 1 | 2.45 |

After each step the mixture was homogenized by gentle stirring. The droplet size of the resulting emulsion of Comparative Example A was measured with a Mastersizer S, using the presentation code 3OHD. The d50 of this emulsion was 220 nm.

The emulsions of Examples 1 and 2 were prepared starting from the emulsion of Comparative Example A. The emulsion of Example 1 was obtained by adding 0.5 wt % of a silica (Bindzil Cat 80 ex Eka) to the starting emulsion, based on the weight of the oil. The emulsion of Example 2 was obtained by adding 1 wt % of a silica (Bindzil Cat 80 ex Eka) to the starting emulsion, based on the weight of the oil.

All three emulsions were placed in an oven at 54° C. The emulsion of Comparative Example A showed a clear oil layer within eight days of storage. The emulsions of Examples 1 and 2 did not show a clear oil layer and remained homogeneous even after 30 days of storage.

This clearly demonstrates that the stability against coalescence at elevated temperature is improved due to the addition of the silica. Consequently, the storage life time is also considerably improved for emulsions in accordance with the present invention.

### Examples 3 and 4 and Comparative Example B

At room temperature an oil-in-water emulsion comprising a methyl ester of rape-seed oil, demineralized water, di/tristyrylphenol ethoxylate (15EO), and sodium bis(2-ethylhexyl) sulfosuccinate was prepared in three steps as illustrated in the Table 2 below.

TABLE 2

| | step I | Step II | Step III |
|---|---|---|---|
| Ingredients | (g) | (g) | (g) |
| Methyl ester of rape-seed oil | 300 | | |
| Sodium bis(2-ethylhexyl) sulfosuccinate | 52.8 | | |
| Water | | 300 | 834 |
| di/tristyrylphenol ethoxylate (15EO) | | 13.2 | |

After each step the mixture was homogenized by gentle stirring. After Step II the mixture was mixed until it was almost transparent.

The resulting emulsion represents Comparative Example B and the d50 of this emulsion was 100 nm as measured with a Malvern Zetasizer.

TABLE 3

| | Step I | Step II |
|---|---|---|
| Ingredients | (g) | (g) |
| Water | 196 | |
| Laponite RD ex Rockwood | 4 | |
| Bindzil ® CAT 80 ex AkzoNobel | | 1.28 |

A colloidal solution is made in two steps as illustrated in Table 3. After Step I the mixture is stirred until it is almost transparent. The Bindzil® CAT 80 is added dropwise into the mixture during stirring.

The emulsions of Examples 3 and 4 were prepared starting from the emulsion of Comparative Example B. The emulsion of Example 3 was obtained by adding 4.82 grams of the colloidal solution described above to the 20 grams of the starting emulsion of comparative example B. The emulsion of Example 4 was obtained by adding 7.8 grams to 20 grams of the colloidal solution described above to the starting emulsion, of comparative example B.

All three emulsions were placed in an oven at 54° C. The emulsion of Comparative Example B showed a water layer within four days of storage. The emulsions of Examples 3 and 4 did not show any separation and remained homogeneous even after 14 days of storage.

This clearly demonstrates that the stability against coalescence at elevated temperature is improved due to the addition of the silica/clay mixture. Consequently, the storage lifetime is also considerably improved for emulsions in accordance with the present invention.

### Example 5 and Comparative Example C

At room temperature an oil-in-water emulsion comprising 28.7 parts by weight of cetearyl isononanoate, 65 parts by weight of demineralized water, 4.2 parts by weight of nonionic emulsifier C₁₆₋₁₈EO12, and 2.1 parts by weight of glyceryl monostearate was prepared. While stirring, the mixture was heated to 90° C. and cooled down to room temperature again. During this process the conductivity was followed. A sharp decrease of the conductivity was measured in the range of 70-80° C., marking the transition from an oil-in-water emulsion to a water-in-oil emulsion (i.e. the phase inversion). The emulsion was subsequently cooled down gradually to room temperature, thereby rendering an oil-in-water emulsion. The d50 value of the droplets was 112 nm. The obtained emulsion is Comparative Example C which is not in accordance with the invention.

To part of the obtained emulsion a silica sol (Bindzil 257/360 ex AkzoNobel) was added in an amount of 20 wt % based on the weight of the oil droplets. The obtained emulsion was in accordance with the invention (Example 5). The d50 value of the droplets was 125 nm.

The emulsions of Example 5 and Comparative Example C were both placed in an oven at 60° C. The particle size was measured after 10 days. The emulsion of Comparative Example C contained oil droplets having a d50 value of 590 nm and revealed visual separation (also referred to as creaming). The emulsion of Example 5 contained oil droplets having a d50 value of 118 nm; this emulsion was homogeneous and no separation was observed.

This demonstrates that the emulsion of the invention has a much better storage lifetime at elevated temperatures than the emulsion of Comparative Example C.

1. A stable emulsion comprising:
(a) an oil;
(b) water;
(c) a surfactant; and
(d) solid particulate material,
wherein an emulsion of just (a), (b), and (c) has a transitional phase inversion point, and preferably a phase inversion temperature, wherein the stable emulsion contains droplets having a d50 value of below 1 μm, wherein the emulsions comprising components (a) to (d) has a longer shelf life time than emulsions containing only compounds (a) to (c), when stored at the same temperature, and wherein preferably the change in d50 value of the emulsion comprising compounds (a) to (d) is lower than the d50 value of emulsions containing only compounds (a) to (c) during storage, with the proviso that the emulsion is not an oil in water emulsion whereof at least 25% by weight of the oil has a molecular weight greater than 400 Dalton and whereof the oil/surfactant ratio is 0.8-3.5.

2. Emulsion according to claim 1 wherein the droplets have a d90 value of 5 μm or less.

3. Emulsion according to claim 1 wherein the droplets comprise oil.

4. Emulsion according to claim 1 wherein the droplets comprise water.

5. Emulsion according to claim 1 wherein the stable emulsion has a phase inversion temperature which is higher than the phase inversion temperature of a similar emulsion containing only compounds (a) to (c).

6. Emulsion according to claim 1 wherein the average particle size of the solid particulate material is at least five times lower, preferably at least 10 times lower, than the d50 value of the droplets in the stable emulsion.

7. Emulsion according to claim 1 wherein the solid particulate material is cationic and/or anionic, preferably anionic.

8. Emulsion according to claim 1 wherein the solid particulate material is an inorganic oxygen-containing particulate material.

9. A process for preparing the stable emulsion according to claim 1 comprising the steps of:
(a) contacting water, oil, and a surfactant so as to obtain an emulsion having a transitional phase inversion; and
(b) causing the emulsion to have a phase inversion so as to form a stable emulsion comprising droplets having a d50 value of below 1 um; and
wherein a solid particulate material is added to the emulsion before, during and/or after the phase inversion of step (b).

10. Process according to claim 9 wherein the phase inversion of step (b) is invoked by a change in composition and/or a change in temperature.

11. An emulsion obtainable by the process of claim 9.

12. The emulsion of claim 1 comprising an agro-chemical.

13. A method of delivering an agro-chemical to a crop which comprises contacting said crop with the emulsion of claim 12.

14. The emulsion of claim 1 comprising a cosmetically-active ingredient.

15. A method of delivering a cosmetically-active ingredient to the skin which comprises contacting said skin with the emulsion of claim 14.

16. Emulsion according to claim 1 wherein the droplets have a d90 value of 1 μm or less.
